(19) United States
(12) Patent Application Publication
(10) Pub. No.: US 2007/0243558 A1
(43) Pub. Date: Oct. 18, 2007

(54) Peptide Stabilization

(76) Inventors: Osmo Suovaniemi, Helsinki (FI); Auli Linanla, Helsinki (FI)

Correspondence Address: BIRCH STEWART KOLASCH & BIRCH, PO BOX 747, FALLS CHURCH, VA, 22040-0747, US

(21) Appl. No.: 11/579,004

(22) PCT Filed: May 9, 2005
(86) PCT No.: PCT/FI05/50147

§ 371 Date: Oct. 30, 2006

(30) Foreign Application Priority Data
May 10, 2004 (FI) 20040655

(51) Int. Cl. G01N 33/68; C07C 323/03

(52) U.S. Cl. 435/7.1; 568/23

(57) ABSTRACT

The present invention is directed to the use of DMSO for the stabilization of temperature sensitive proteins and peptides, such as gastrin, against degradation in a mammalian biological sample, such as a serum or plasma sample.

### FIELD OF THE INVENTION

The present invention generally relates to the field of stabilization of temperature sensitive analytes, especially proteins and peptides, including hormones and enzymes, against degradation, in biological samples, such as plasma or serum samples from mammals.

### BACKGROUND OF THE INVENTION

One problem experienced in connection with assaying analytes in biological samples, especially in serum and plasma samples, relates to the poor stability of some proteins and peptides. Small peptides are especially sensitive to degradation by enzymes. The poor stability requires that the sample is tested immediately, or is stored in a cold environment, such as a refrigerator. However, even under cool storage the stability is often insufficient. If such samples are to be stored over many days, they have to be packed in dry ice. This is naturally a considerable inconvenience.

Gastrin-17 is a peptide hormone which is secreted by the G-cells of the antrum of the stomach under the stimulation. The stimulation is the result of breakdown of the proteins after a protein rich meal. Also stretching of the stomach gives rise to a secretion of gastrin, especially gastrin-17.

The gastrin peptides are formed from one pro-gastrin molecule. Most peptide hormones are synthetized by subjecting pro-hormones to proteolytic cleavage and enzymatic modifications. Gastrin processing can be divided into three categories: pro-gastrins, glycine-extended derivatives and carboxy-amidated gastrins. Several different biologically active gastrin forms can be isolated from the human stomach, of which the most important are gastrin-34 and gastrin-17. In addition, gastrin-71 has been isolated from plasma, which is the largest bioactive gastrin, as well as gastrin-52 and gastrin-14.

Gastrin-17 is the strongest hydrochloric acid stimulant. Gastrin-17 is present in two forms, in a non-sulfonated form (gastrin I) and in a sulfonated form, wherein tyrosine is sulfonated (gastrin II). Both of these forms are equally effective. The active form of gastrin-17 is the amidated gastrin-17, wherein the last amino acid, phenyl alanine of the peptide, contains an extra amide group. The non-active form is the so-called glycine extended gastrin-17.

Various methods and kits are known for use in determining the condition of the mucous membrane of the stomach in an individual. Based on such measurements, it is possible to determine i.a. a risk for the individual to develop cancer of the stomach. Examples of such methods are described for example in the following patents and patent applications: EP 804 737, EP 990992, EP 02716100, and PCT/FI03/000653 of the present applicant.

In these methods, commonly at least the following analytes or markers are determined from a sample, such as a serum or plasma sample form an individual: pepsinogen I, gastrin-17 and a marker for Helicobacter pylori infection, and, in addition, often pepsinogen II.

In connection with said determinations and sample handling the poor stability of gastrin-17 has proven to be a problem, due to the fact that it decomposes rapidly in the sample. After three hours, the gastrin-17 concentration in a serum sample starts to decrease clearly and after a day it has degraded completely under refrigerating conditions. This is in contrast to pepsinogen I and II, which retain their activity at room temperature for many days, and Helicobacter pylori antibodies, which survive for at least three days under refrigerator conditions.

Gastrin-17 has previously been taken as a so-called cold sample, that is the coagulation of the serum sample is done on an ice bath. Delivery or dispatch of the sample extending over many days has been done in frozen form.

Due to the problems encountered with the unsatisfactory stability of not only gastrin type peptides, but equally of a number of other proteins and peptides, including hormones and enzymes, efforts have been made in finding a means of stabilizing these sensitive analytes. The minimum requirement for a satisfactory stabilization for the purpose of acceptable laboratory procedures is a stability of at least three days both at room or ambient temperature and refrigerator temperature, that is at a temperature range of appr. +4° C. to 25° C.

### SUMMARY OF THE INVENTION

According to the invention it has now been discovered that dimethyl sulfoxide (DMSO) is a very efficient stabilizer for proteins and peptides, especially gastrin and gastrin related peptides, in a mammalian biological sample, especially in a serum or plasma sample, providing a stabilizing effect for said proteins and peptides which lasts for at least three days both at ambient and refrigerator temperatures.

Thus the invention is on the one hand directed to the use of DMSO for the stabilization of gastrin and gastrin related peptides, against degradation or inactivation in a mammalian biological sample, such as a serum or plasma sample. On the other hand, the invention is also directed to a method for stabilizing such peptides, against degradation or inactivation in a mammalian biological sample, such as a serum or plasma sample, according to which method a stabilizing amount of DMSO is added to the sample containing the said peptide to be stabilized.

### DETAILED DESCRIPTION OF THE INVENTION

The stabilizer to be used according to the invention is an organic solvent, dimethyl sulfoxide, DMSO, well known for a variety of industrial, medical and laboratory uses. The molecular mass of DMSO is 78.1, it is miscible with water, has a melting point of 18.5° C. and a boiling point of 189° C. It is a by-product from the paper industry, wherein it is derived from the lignin, the binding substance in trees.

In cell cultivation DMSO is routinely used as a 10% solution when freezing cells. In this application DMSO acts as a protectant reducing the osmotic damage, as cells are very sensitive to ice formation within the cells. DMSO penetrates rapidly the cell membrane and replaces the water therein. Consequently, DMSO also penetrates the skin rapidly and is able to easily transport through the skin a component dissolved therein, such as a low molecular component. For this reason DMSO is used as an adjuvant promoting the absorption and transport of drug molecules. DMSO has also been reported as having a wide variety of pharmaceutical applications, including reduction of pain and swelling, muscle relaxation, anti-inflammatory and bacteriostatic properties etc.

According to the invention DMSO is contemplated for use for stabilizing temperature sensitive peptides, i.e. gastrin and gastrin related peptides, which are susceptible to rapid decomposition within a period of a few, such as within three days, in a biological sample, even when kept under refrigerating conditions.

Especially useful is the present invention for the stabilization of gastrins and gastrin related peptides, such as cholecystocinins and secretins, and especially for the stabilization of gastrin-17. The invention is especially applicable for use in a method, e.g. in a test for screening the condition of the gastric mucosa, wherein gastrin-17 is to be determined together with the analytes pepsinogen I and optionally pepsinogen II, as well as a marker for Helicobacter pylori from a biological sample, such as a serum, plasma sample.

During the course of experimentation it has been proven that DMSO added to such samples exert a stabilizing effect on gastrin, without having an adverse effect on the other markers to be assayed.

The amount of DMSO to be used for obtaining a stabilizing effect can vary, but is typically of the order of 1 to 10% (vol./vol.), especially appr. 4 to 6% (vol./vol.) calculated from the sample to be tested.

In the context of the invention ‘temperature sensitive’ in connection with an analyte peptide in a mammalian biological sample means that the said peptide is prone to rapid degradation especially at room or ambient temperature. A temperature sensitive analyte to be stabilized according to the invention is thus one that has a storage time of less than 3 days (72 hours) at room or ambient temperature, typically being a temperature range of appr. +18 to 25° C.

According to the invention an amount of DSMO so added to a mammalian biological sample to be stabilized provides a stability for the analyte peptide which is at least three days (at least 72 hours) at the above mentioned temperature range.

Experimental

Experiments carried out in the course of investigations relating to the stability of gastrin-17 in plasma and serum samples have shown that gastrin-17 was preserved in 5% (vol./vol.) DMSO/serum or plasma solutions for at least three days even at room temperature. This is evident from the test results presented in the following tables.

Table 1 describes the results from tests wherein three EDTA-plasma samples were tested as to gastrin-17 stability during a time period of up to three days by adding 5% (vol./vol.) DMSO to said samples. The reference examples had no added DMSO. The results show an improved stability of gastrin-17 after 3 days in the samples containing DMSO, as compared to the samples containing no DMSO.

Table 2 shows the stabilizing effect of adding DMSO to five EDTA-plasma samples at room temperature. The effect of adding DMSO was tested on pepsinogen I, pepsinogen II, Helicobacter pylori antibodies and gastrin-17. The results are indicated at day 0 and day 3. The results show that there is essentially no effect on the activity of pepsinogen I and II, and Helicobacter pylori antibodies, whereas an improvement is observed for the stability of gastrin-17 as compared to the results obtained for the samples with no added DMSO.

Table 3 is a test corresponding to the test of Table 2, but where the ten samples to be tested were serum samples rather than plasma samples. The results show a generally retained activity for gastrin-17 in the samples with added 5% (vol./vol.) of DMSO. The results also show that the DMSO does not have a detrimental effect on the activities of pepsinogens and H. pylori antibodies

Table 4 shows the results of stability tests of samples where the analyte was U-INTP (collagen I aminoterminal telopeptide). The table shows the results immediately (“immed.”) and after three days at room temperature without and with stabilizer, expressed also as percentages. Two parallel tests were done for each sample.

TABLE 1

| | PL459S | PL459S | PL460s | PL460s | PL461s | PL461s |
|---|---|---|---|---|---|---|
| | 0% DMSO | 5% DMSO | 0% DMSO | 5% DMSO | 0% DMSO | 5% DMSO |
| Days | pmol/l | pmol/l | pmol/l | pmol/l | pmol/l | pmol/l |
| 0 | 8.5 | 6.3 | 7.9 | 6.3 | 26.0 | 24.3 |
| 1 | 3.6 | 5.4 | 5.8 | 5.5 | 8.7 | 21.6 |
| 2 | 2.7 | 5.2 | 5.8 | 6.1 | 8.5 | 19.9 |
| 3 | 1.5 | 5.4 | 4.6 | 6.0 | 6.1 | 21.2 |
| Avg. | 4.1 | 5.6 | 6.0 | 6.0 | 12.4 | 21.8 |
| STDEV | 3.09 | 0.52 | 1.37 | 0.32 | 9.20 | 1.83 |
| CV % | 75.7 | 9.3 | 22.7 | 5.3 | 74.4 | 8.4 |

TABLE 2
PGI

| | μg/l | μg/l | | | |
|---|---|---|---|---|---|
| | 0% DMSO | 5% DMSO | | | |
| days | PL466s | PL466s | avg. | stdev | cv % |
| 0 | 58.8 | 60.5 | 59.65 | 1.2 | 2.0 |
| 3 | 76.9 | 77.4 | 77.15 | 0.4 | 0.5 |
| | PL467s | PL467s | | | |
| 0 | 73.3 | 76.8 | 75.05 | 2.5 | 3.3 |
| 3 | 94.9 | 98.8 | 96.85 | 2.8 | 2.8 |
| | PL471s | PL471s | | | |
| 0 | 60.6 | 67.6 | 64.1 | 4.9 | 7.7 |
| 3 | 85.5 | 84.3 | 84.9 | 0.8 | 1.0 |
| | PL472s | PL472s | | | |
| 0 | 110.3 | 109.4 | 109.85 | 0.6 | 0.6 |
| 3 | 134.8 | 135.6 | 135.2 | 0.6 | 0.4 |
| | PL473s | PL473s | | | |
| 0 | 94.9 | 94.7 | 94.8 | 0.1 | 0.1 |
| 3 | 115.4 | 102.1 | 108.75 | 9.4 | 8.6 |

PGII

| | μg/l | μg/l | | | |
|---|---|---|---|---|---|
| | 0% DMSO | 5% DMSO | | | |
| days | PL466s | PL466s | avg. | stdev | cv % |
| 0 | 6.8 | 6.6 | 6.7 | 0.1 | 2.1 |
| 3 | 8.3 | 7.7 | 8 | 0.4 | 5.3 |
| | PL467s | PL467s | | | |
| 0 | 5 | 4.9 | 4.95 | 0.1 | 1.4 |
| 3 | 6.4 | 6.2 | 6.3 | 0.1 | 2.2 |
| | PL471s | PL471s | | | |
| 0 | 4.9 | 4.8 | 4.85 | 0.1 | 1.5 |
| 3 | 5.8 | 5.3 | 5.55 | 0.4 | 6.4 |
| | PL472s | PL472s | | | |
| 0 | 5.7 | 5.7 | 5.7 | 0.0 | 0.0 |
| 3 | 7.3 | 6.9 | 7.1 | 0.3 | 4.0 |
| | PL473s | PL473s | | | |
| 0 | 6.4 | 6.4 | 6.4 | 0.0 | 0.0 |
| 3 | 8.5 | 7.9 | 8.2 | 0.4 | 5.2 |

H. pylori

| | EIU | EIU | | | |
|---|---|---|---|---|---|
| | 0% DMSO | 5% DMSO | | | |
| days | PL466s | PL466s | avg. | stdev | cv % |
| 0 | 6.3 | 8.6 | 7.45 | 1.6 | 21.8 |
| 3 | 6.5 | 6.4 | 6.45 | 0.1 | 1.1 |
| | PL467s | PL467s | | | |
| 0 | 10.3 | 10.9 | 10.6 | 0.4 | 4.0 |
| 3 | 8 | 7.3 | 7.65 | 0.5 | 6.5 |
| | PL471s | PL471s | | | |
| 0 | 7 | 6.8 | 6.9 | 0.1 | 2.0 |
| 3 | 7.4 | 7 | 7.2 | 0.3 | 3.9 |
| | PL472s | PL472s | | | |
| 0 | 47.2 | 45.7 | 46.45 | 1.1 | 2.3 |
| 3 | 52 | 50.4 | 51.2 | 1.1 | 2.2 |
| | PL473s | PL473s | | | |
| 0 | 3.5 | 3.7 | 3.6 | 0.1 | 3.9 |
| 3 | 3.5 | 3.9 | 3.7 | 0.3 | 7.6 |

Gastrin 17

| | pmol/l | pmol/l | | | |
|---|---|---|---|---|---|
| | 0% DMSO | 5% DMSO | | | |
| days | PL466s | PL466s | avg. | stdev | cv % |
| 0 | 7.6 | 7.8 | 7.7 | 0.1 | 1.8 |
| 3 | 1.6 | 6.9 | 4.25 | 3.7 | 88.2 |
| | PL467s | PL467s | | | |
| 0 | 5.3 | 4.4 | 4.85 | 0.6 | 13.1 |
| 3 | 2.9 | 4.5 | 3.7 | 1.1 | 30.6 |
| | PL471s | PL471s | | | |
| 0 | 1.3 | 1.7 | 1.5 | 0.3 | 18.9 |
| 3 | 0.2 | 1.4 | 0.8 | 0.8 | 106.1 |
| | PL472s | PL472s | | | |
| 0 | 20 | 21 | 20.5 | 0.7 | 3.4 |
| 3 | 2.9 | 14.3 | 8.6 | 8.1 | 93.7 |
| | PL473s | PL473s | | | |
| 0 | 3.7 | 3.8 | 3.75 | 0.1 | 1.9 |
| 3 | 0.6 | 3.4 | 2 | 2.0 | 99.0 |

TABLE 3

| | 0% DMSO | 5% DMSO | avg. | st.dev. | cv % |
|---|---|---|---|---|---|
| | H. pylori | | | | |
| | EIU | | | | |
| PL411s | 120.2 | 107.8 | 114.0 | 8.8 | 7.7 |
| PL420b | 19.0 | 18.5 | 18.8 | 0.4 | 1.9 |
| PL426s | 48.5 | 43.2 | 45.9 | 3.7 | 8.2 |
| PL438s | 2.9 | 1.6 | 2.3 | 0.9 | 40.9 |
| PL439s | 17.9 | 14.4 | 16.2 | 2.5 | 15.3 |
| PL447b | 16.0 | 13.6 | 14.8 | 1.7 | 11.5 |
| PL454s | 24.0 | 19.5 | 21.8 | 3.2 | 14.6 |
| PL455s | 23.0 | 20.0 | 21.5 | 2.1 | 9.9 |
| PL457b | 4.8 | 4.3 | 4.6 | 0.4 | 7.8 |
| PL458s | 58.9 | 51.1 | 55.0 | 5.5 | 10.0 |
| | PGI | | | | |
| | μg/l | | | | |
| PL411s | 283.6 | 296.0 | 289.8 | 8.8 | 3.0 |
| PL420b | 75.5 | 79.3 | 77.4 | 2.7 | 3.5 |
| PL426s | 49.7 | 50.6 | 50.2 | 0.6 | 1.3 |
| PL438s | 158.6 | 156.0 | 157.3 | 1.8 | 1.2 |
| PL439s | 67.6 | 68.5 | 68.1 | 0.6 | 0.9 |
| PL447b | 6.0 | 6.0 | 6.0 | 0.0 | 0.0 |
| PL454s | 100.6 | 102.3 | 101.5 | 1.2 | 1.2 |
| PL455b | 79.2 | 81.5 | 80.4 | 1.6 | 2.0 |
| PL457b | 101.1 | 106.9 | 104.0 | 4.1 | 3.9 |
| PL458s | 77.1 | 80.1 | 78.6 | 2.1 | 2.7 |
| | PGII | | | | |
| | μg/l | | | | |
| PL411s | 32.8 | 33.6 | 33.2 | 0.6 | 1.7 |
| PL420b | 6.3 | 5.7 | 6 | 0.4 | 7.1 |
| PL426s | 3.5 | 3.1 | 3.3 | 0.3 | 8.6 |
| PL438s | 13.6 | 12.8 | 13.2 | 0.6 | 4.3 |
| PL439s | 5.7 | 5.2 | 5.45 | 0.4 | 6.5 |
| PL447b | 4.9 | 4.6 | 4.75 | 0.2 | 4.5 |
| PL454s | 7.7 | 7.3 | 7.5 | 0.3 | 3.8 |
| PL455b | 7.3 | 7.1 | 7.2 | 0.1 | 2.0 |
| PL457b | 7.1 | 6 | 6.55 | 0.8 | 11.9 |
| PL458s | 6.7 | 5.8 | 6.25 | 0.6 | 10.2 |
| | Gastrin 17 | | | | |
| | pmol/l | | | | |
| PL411s | 33.4 | 32.5 | 33.0 | 0.6 | 1.9 |
| PL420b | 24.1 | 20.1 | 22.1 | 2.8 | 12.8 |
| PL426s | 1.1 | 1.9 | 1.5 | 0.6 | 37.7 |
| PL438s | 9.3 | 9.8 | 9.6 | 0.4 | 3.7 |
| PL439s | 7.4 | 8.5 | 8.0 | 0.8 | 9.8 |
| PL447b | 18.2 | 21.7 | 20.0 | 2.5 | 12.4 |
| PL454s | 4.4 | 5.6 | 5.0 | 0.8 | 17.0 |
| PL455b | 15.6 | 17.6 | 16.6 | 1.4 | 8.5 |
| PL457b | 3.3 | 6.0 | 4.7 | 1.9 | 41.1 |
| PL458s | 5.4 | 2.2 | 3.8 | 2.3 | 59.5 |

TABLE 4

| | | | in % of | | in % of |
|---|---|---|---|---|---|
| | | 3 days no | “immed.”- | 3 days + | “immed.”- |
| Test: | Immed. | stabilizer | sample | stabilizer | sample |
| U-INTP | | | | | |
| Sample 1 | 200 | 189 | 94.5 | 202 | 101.0 |
| Sample 1 | 199 | 187 | 94.0 | 209 | 105.0 |
| parall. | | | | | |
| Sample 2 | 113 | 115 | 101.8 | 144 | 127.4 |
| Sample 2 | 118 | 114 | 96.6 | 136 | 115.3 |
| parall. | | | | | |

1. Use of DMSO for the stabilization of gastrin and gastrin related peptides against degradation in a mammalian biological sample.

2. The use according to claim 1, wherein the sample is a serum or plasma sample.

3. The use according to claim 1 or 2 for the stabilization of gastrin-17.

4. Method for stabilizing gastrin and gastrin related peptides peptides against degradation in a mammalian biological sample, according to which method a stabilizing amount of DMSO is added to the said sample.

5. The method according to claim 4 wherein the sample is a serum or plasma sample.

6. The method according to claim 4 or 5 for the stabilization of gastrin-17.

7. The method according to claim 6, wherein gastrin-17 is determined in a serum or plasma sample containing one or more analytes selected from the group consisting of pepsinogen I, pepsinogen II and Helicobacter pylori antibodies.
